(19) United States
(12) Patent Application Publication
(10) Pub. No.: US 2019/0300911 A1
(43) Pub. Date: Oct. 3, 2019

(54) METHODS FOR INCREASING THE FREQUENCY OF GENE TARGETING BY CHROMATIN MODIFICATION

(71) Applicant: FRIEDRICH MIESCHER INSTITUTE FOR BIOMEDICAL RESEARCH, Basel (CH)

(72) Inventors: Susan M. GASSER, Lausanne (CH); Andrew SEEBER, Basel (CH); Michael Hermann HAUER, Lörrach (DE)

(21) Appl. No.: 16/346,752

(22) PCT Filed: Nov. 1, 2017
(86) PCT No.: PCT/IB2017/056793

§ 371 Date: May 1, 2019

(30) Foreign Application Priority Data
Nov. 3, 2016 (EP) 16197064.5

(51) Int. Cl. C12N 15/90; C07K 14/47; C12N 15/10

(52) U.S. Cl. CPC C12N 15/907; C07K 14/47; C12N 2800/00; C12N 2510/00; C12N 15/1024

(57) ABSTRACT

The present disclosure relates to methods and kits for increasing the frequency of insertion of a nucleotide sequence of interest in a specific chromosomal site within the genome of a eukaryotic cell by modifying the state of chromatin in said cell. In the present disclosure chromatin state may be altered by reducing nucleosomal occupancy by modulating the activity of high-mobility group box (HMGB) proteins.

### TECHNICAL FIELD

The present disclosure relates to a novel method of increasing the frequency of insertion of a sequence of interest in a specific chromosomal site by modifying the state of chromatin of a eukaryotic cell.

### BACKGROUND OF THE INVENTION

Gene targeting is a technique to introduce genetic change into specific locations in the genome of a cell. The targeted introduction of genetic changes can be used as a powerful experimental approach. The technique can be used to introduce essentially any desirable change in a genomic sequence, including the introduction of novel sequences, such as transgenes for expression, the inactivation or attenuation of a gene, or the introduction of a sequence change that confers an improved phenotype. It can be used to ameliorate a genetic disorder in a subject, to confer a desirable genotype on a subject or cell, to increase the production or activity of a beneficial polypeptide in a subject or cell, to decrease the production or activity of an undesirable polypeptide in a subject or cell and to investigate the effects of genetic changes in a non-human organism or any cell type. In gene targeting experiments, the exchange of genetic information is promoted between an endogenous chromosomal sequence and an exogenous DNA construct and the nucleotide sequence at a predetermined genomic site is selectively altered by introduction of an exogenous nucleic acid carrying a desired sequence. Depending of the design of the targeted construct, genes can be knocked out, knocked in, replaced, corrected or mutated, in a rational, precise and efficient manner.

A eukaryotic genome exists in the nucleus of a cell as a complex of protein and double stranded DNA (dsDNA) termed chromatin. The packaging of a mammalian genome into chromatin serves to compact and organize the genome, and also regulates the accessibility of the dsDNA.

The basic repeating unit of chromatin is termed the nucleosome. A nucleosome is a complex of at least a core of eukaryotic (e.g., mammalian) histone proteins (e.g., two H2A proteins, two H2B proteins, two H3 proteins, and two H4 proteins) with about 147 base pairs of a dsDNA molecule wrapped around the core of eukaryotic (e.g., mammalian) histone proteins.

Chromatin structure is not static, but is subject to modification by processes collectively known as chromatin remodeling. Chromatin remodeling can serve, for example, to remove nucleosomes from a region of DNA, move nucleosomes from one region of DNA to another, change the spacing between nucleosomes or add or remove nucleosomes to a region of DNA in the chromosome. This positioning and occupancy of nucleosomes results in chromatin remodeling that can result in changes in higher order structure, thereby influencing the balance between transcriptionally active chromatin (open chromatin or euchromatin) and transcriptionally inactive chromatin (closed chromatin or heterochromatin). Because nucleosomes inhibit the access of other DNA-binding proteins to DNA, nucleosome positioning and occupancy are therefore critical factors to biological outcomes.

The gene targeting process requires some degree of homology between the targeting construct and the targeted locus and it is significantly stimulated by free DNA ends in the construct (Orr-Weaver et al. 1981, Orr-Weaver and Szostak 1983, Szostak et al. 1983). The free DNA ends label the construct as a substrate for the Homologous Recombination (HR) machinery, a very conserved DNA maintenance pathway involved in the repair of DSBs and other DNA lesions (Paques and Haber 1999, Sung and Klein 2006) that promotes the exchange of genetic information between endogenous sequences. The frequency of HR can be significantly increased by a specific DNA double-strand break (DSB) at a chromosomal site of interest (Rouet et al. 1994, Choulika et al. 1995). Local chromatin structure plays an important role in the positioning and frequency of double-strand breaks leading to homologous recombination (Ohta et al. 1994).

Although powerful tools are available for the manipulation of the eukaryotic genome, there is a need to increase the efficiency of gene targeting, i.e. the frequency of integration events of an exogenous nucleic acid at a targeted locus. Nucleosome packing and chromatin architecture surrounding the double strand break may limit the ability of the DNA damage response to access and repair the break thereby impeding insertion of a sequence of interest at a target site (Price and D'Andrea 2013). In higher organisms, and in mammalian cells in particular, only very low frequencies of targeted events have been achieved, usually in the range of 10⁻⁶ per cell (Doetschman et al. 1988). In addition, gene targeting occurs against a background of non-homologous events that are 100- to 1000-fold more common (Mansour et al. 1988), meaning that the exogenous nucleic acid sequence is inserted at non-selected positions on the genome (“off-target insertions”).

### SUMMARY OF THE INVENTION

The present inventor serendipitously found that by modifying the state of chromatin in a eukaryotic cell, and in particular by lowering nucleosomal occupancy, the frequency of insertion of a sequence of interest at a specific chromosomal site within a eukaryotic genome can be increased and the frequency of insertion, e.g. by homologous recombination, of the targeting cassette at undesired chromosomal sites within a eukaryotic genome can be drastically decreased.

The present invention thus provides a method for increasing the frequency of insertion of a nucleotide sequence of interest in a specific chromosomal site within the genome of a eukaryotic cell, said method comprising the step of modifying the state of chromatin in said cell. It is to be understood that inserting encompasses the replacement of a sequence by another one, i.e. editing. In some embodiments, the state of chromatin in said cell is modified by lowering nucleosome occupancy. In some embodiments, the nucleosome occupancy is lowered transiently. In some embodiments, the chromatin state is altered by reducing nucleosomal occupancy by modulating (inhibiting or promoting, wherein inhibiting also encompasses modifying the binding of a protein by e.g. competitors) the activity of high-mobility group box (HMGB) proteins. In some embodiments, the HMGB proteins are inhibited by an agent from the group comprising Glycyrrhizin, Tanshinone IIA, Epigallocatechin-3-gallate, Quercetin, Lycopene, nafamostat mesilate, gabexate mesilate, ethyl pyruvate, carbenoxolone, antibodies and Oligonucleotide (ODN)-based inhibitors of HMGB1. In some embodiments, a cassette comprising said nucleotide sequence of interest and at least a first region having sequence identity with said chromosomal site is introduced into said eukaryotic cell and said nucleotide sequence of interest is inserted into the target site. In some embodiments, the insertion event is by a homologous recombination or non-homologous end-joining. In some embodiments, the composition further comprises an agent introducing double-strand breaks (DSB) within the genome. The DSB agent can be selected from the group comprising Transcription activator-like effector nucleases (TALEN), zinc-finger nucleases (ZFN), CRISPR/Cas9, CRISPR/nickase, megaendonucleases, NgAgo and chimeric nucleases. In some embodiments, the double-strand break is at a specific chromosomal site within the genome of a eukaryotic cell.

The present invention also provides a kit comprising an agent lowering nucleosome occupancy in eukaryotic cells and a targeting cassette. In some embodiments, the kit comprises an agent inducing double strand breaks in eukaryotic cells. In some embodiments, the kit comprises an HMGB1 inhibitor as an agent lowering nucleosome occupancy in eukaryotic cells. The HMGB1 inhibitor can be selected from the group comprising Glycyrrhizin, Tanshinone IIA, Epigallocatechin-3-gallate, Quercetin, Lycopene, nafamostat mesilate, gabexate mesilate, ethyl pyruvate, carbenoxolone, antibodies and Oligonucleotide (ODN)-based inhibitors of HMGB1. The agent inducing double strand breaks in eukaryotic cells can be selected from the group comprising Transcription activator-like effector nucleases (TALEN), zinc-finger nucleases (ZFN), CRISPR/Cas9, CRISPR/nickase, megaendonucleases, NgAgo and chimeric nucleases.

### BRIEF DESCRIPTION OF THE DRAWINGS

FIG. 1: Reduced nucleosome occupancy enhances recombination. a, Ectopic recombination assay with two different integrative URA3 cassettes in WT, arp8A and nhp6A strains. The diagram on the left highlights that recombination takes place in the context of chromatin. Bar graphs show the integration frequency in selected mutants relative to the WT which was set to 1. b, Ectopic recombination assay with two different hygromycin (hphMX4) based constructs which target either the ATG2 or MGS1 locus. Bar graphs show the integration frequency of both constructs in the SD strain relative to the Ctr. strain after 120 min. pulsed histone H3/H4 reductions in Gal:Raff (galactose:raffinose, 1:20) or raffinose (Raft) medium. The Ctr. strain was set to 1. Bar graphs (a and b) show means±s.e.m. *P<0.05, ***P<0.001, two-tailed paired students T-test. Isogenic WT, wild type.

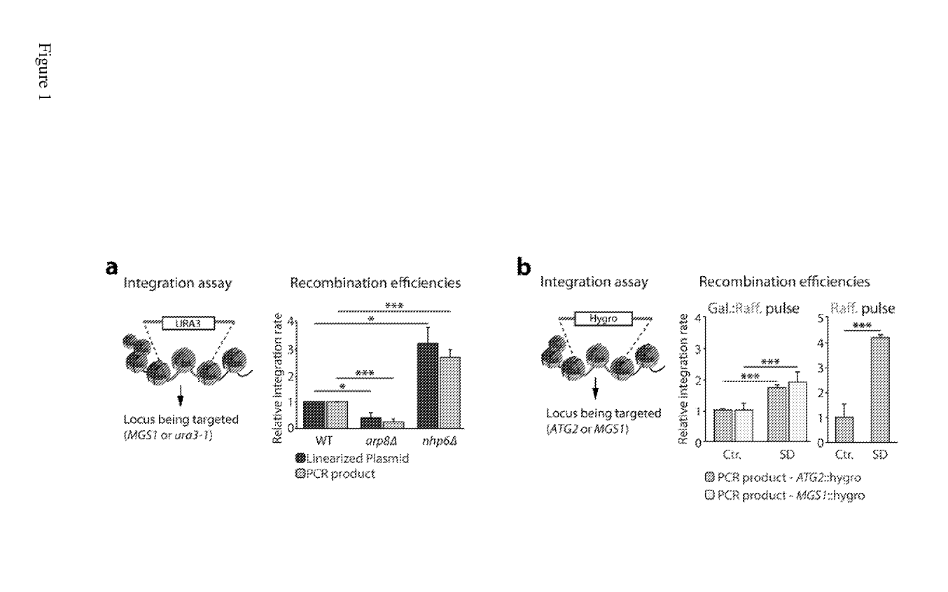

### DETAILED DESCRIPTION

The present invention is based in part on the discovery of methods and compositions for gene targeting in cells, and particularly in mammalian cells, which methods and compositions alter the state of chromatin by lowering the occupancy of nucleosomes such that the frequency of gene targeting events is increased and the frequency of insertion at non-selected positions within the genome is decreased.

The present invention thus provides a method for increasing the frequency of insertion of a nucleotide sequence of interest in a specific chromosomal site within the genome of a eukaryotic cell, e.g. by homologous recombination, said method comprising the step of modifying the state of chromatin in said cell. It is to be understood that inserting encompasses the replacement of a sequence by another one, i.e. editing. In some embodiments, the state of chromatin in said cell is modified by lowering nucleosome occupancy. In some embodiments, the nucleosome occupancy is lowered transiently. In some embodiments, the chromatin state is altered by reducing nucleosomal occupancy by modulating (inhibiting or promoting, wherein inhibiting also encompasses modifying the binding of a protein by e.g. competitors) the activity of high-mobility group box (HMGB) proteins. In some embodiments, the HMGB proteins are inhibited by an agent from the group comprising Glycyrrhizin, Tanshinone IIA, Epigallocatechin-3-gallate, Quercetin, Lycopene, nafamostat mesilate, gabexate mesilate, ethyl pyruvate, carbenoxolone, antibodies and Oligonucleotide (ODN)-based inhibitors of HMGB1. In some embodiments, a cassette comprising said nucleotide sequence of interest and at least a first region having sequence identity with said chromosomal site is introduced into said eukaryotic cell and said nucleotide sequence of interest is inserted into the target site. In some embodiments, the insertion event is by a homologous recombination or non-homologous end-joining. In some embodiments, the composition further comprises an agent introducing double-strand breaks (DSB) within the genome. The DSB agent can be selected from the group comprising Transcription activator-like effector nucleases (TALEN), zinc-finger nucleases (ZFN), CRISPR/Cas9, CRISPR/nickase, megaendonucleases, NgAgo and chimeric nucleases. In some embodiments, the double-strand break is at a specific chromosomal site within the genome of a eukaryotic cell.

The present invention also provides a kit comprising an agent lowering nucleosome occupancy in eukaryotic cells and a targeting cassette. In some embodiments, the kit comprises an agent inducing double strand breaks in eukaryotic cells. In some embodiments, the kit comprises an HMGB1 inhibitor as an agent lowering nucleosome occupancy in eukaryotic cells. The HMGB1 inhibitor can be selected from the group comprising Glycyrrhizin, Tanshinone IIA, Epigallocatechin-3-gallate, Quercetin, Lycopene, nafamostat mesilate, gabexate mesilate, ethyl pyruvate, carbenoxolone, antibodies and Oligonucleotide (ODN)-based inhibitors of HMGB1. The agent inducing double strand breaks in eukaryotic cells can be selected from the group comprising Transcription activator-like effector nucleases (TALEN), zinc-finger nucleases (ZFN), CRISPR/Cas9, CRISPR/nickase, megaendonucleases, NgAgo and chimeric nucleases

In certain embodiments, the present invention provides methods and compositions for increasing the frequency of insertion of a nucleotide sequence of interest in a specific chromosomal site within the genome of a eukaryotic cell by modifying the state of chromatin in said cell. In one embodiment, said cell is a mammalian cell. The cells as used herein, include cultured cells and cell lines. The cell can be an ex vivo cell (e.g., outside an animal's body), or an in vivo cell (e.g., inside an animal's body). The cell can be obtained commercially or from a depository or obtained directly from an individual, such as by biopsy. The cells can be obtained from an individual in need, to whom the cells will be reintroduced once the cells are modified in vitro. Alternatively, the cells can be obtained from another different individual (donor) of the same or different species. For example, nonhuman cells, such as pig cells, can be modified in vitro to include a DNA construct and then introduced into a human. In other cases, the cells need not be isolated from an individual where, for example, it is desirable to deliver the vector to cells of the individual for in vivo gene therapy.

In some embodiments, the method can be used to modify a target sequence. In another embodiment, the method can be used to repair a target sequence. In another embodiment, the method can be used to attenuate or inactivate a target sequence/gene. In a further specific embodiment, the method can used to introduce a heterologous sequence into a site of interest in the chromosome. In certain embodiments, said cell is ex vivo. In certain embodiments, said cell is derived from a cell line.

In one embodiment of the invention, the frequency of insertion of a nucleotide sequence of interest in a specific chromosomal site within the genome of a eukaryotic cell is increased by modifying the state of chromatin comprising lowering nucleosomal occupancy comprising modulating the activity of high-mobility group box (HMGB) proteins.

The term “nucleosomal occupancy” is well known to the skilled person. In accordance, as used herein, the term “nucleosomal occupancy” refers to the density of nucleosomes occupying DNA, or the amount of nucleosomes on a given portion of DNA

As used herein, the term “transiently” refers to the lowering of nucleosomal occupancy which is not permanently lowered. Accordingly, nucleosomal occupancy is lowered before a gene targeting event has taken place and nucleosomal occupancy returns to the state before being lowered after gene targeting has taken place.

High mobility group box 1 protein (HMGB1) is a non-histone chromosomal protein. As a DNA binding protein, HMGB1 is involved in the maintenance of nucleosome structure and the regulation of gene transcription (Travers 2003). It is also active in DNA recombination and repair (Lange et al. 2008). Several types of High mobility group box proteins (HMGBs) have been identified, for example HMGB1-4 in mammals. HMGBs are non-histone proteins highly abundant in the chromatin structure. HMGBs act as DNA chaperones during nucleosome remodeling by binding to the DNA and facilitating the rate limiting DNA distortion. It is thought that HMGBs function as a versatile nucleosome-unwinding factor by optimizing DNA conformation to enhance transcription, replication, recombination, DNA repair, genomic stability and remodeling (Stros 2010).

Several modulators of HMGB1 have been identified, including Glycyrrhizin or derivatives thereof, Tanshinone IIA or derivatives thereof, Epigallocatechin-3-gallate, Quercetin, Lycopene, nafamostat mesilate, gabexate mesilate, ethyl pyruvate, carbenoxolone, methotrexate, antibodies, for example IA-4 and synthetic oligodeoxynucleotide (ODN)-based inhibitors of HMGB1.

In one embodiment of the invention, a HMGB1 modulator may be used to lower nucleosomal occupancy.

In one embodiment of the invention, said HMGB1 modulator is from the group comprising Glycyrrhizin or derivatives thereof, Tanshinone IIA or derivatives thereof, Epigallocatechin-3-gallate (EGCG), Quercetin, Lycopene, nafamostat mesilate, gabexate mesilate, sivelestat, ethyl pyruvate, carbenoxolone, methotrexate, antibodies, for example IA-4 and synthetic oligodeoxynucleotide (ODN)-based inhibitors of HMGB1.

Higher eukaryotes have evolved multiple pathways for the repair of DSBs in a cell, including homologous recombination (HR) and Non-Homologous End Joining (NHEJ).

As used herein the term “homologous recombination” refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques and Haber 1999). In the method of the present invention, homologous recombination is enabled by the presence of said first and said second flanking element being placed upstream (5′) and downstream (3′), respectively, of said donor DNA sequence each of which being homologous to a continuous DNA sequence within said target sequence.

As used herein the term “non-homologous end joining” (NEHJ) refers to cellular processes that join the two ends of double-strand breaks (DSBs) through a process largely independent of homology. Naturally occurring DSBs are generated spontaneously during DNA synthesis when the replication fork encounters a damaged template and during certain specialized cellular processes, including V(D)J recombination, class-switch recombination at the immunoglobulin heavy chain (IgH) locus and meiosis. In addition, exposure of cells to ionizing radiation (X-rays and gamma rays), UV light, topoisomerase poisons or radiomimetic drugs can produce DSBs. NHEJ (non-homologous end-joining) pathways join the two ends of a DSB through a process largely independent of homology. Depending on the specific sequences and chemical modifications generated at the DSB, NHEJ may be precise or mutagenic (Lieber 2010).

Thus, DSBs are a central element of the gene targeting mechanism. Double stranded breaks (cleavages) at a site of interest can be achieved by nucleases or chemical entities which recognize and cleave the site of interest.

As used herein, the term “cleavage” refers to the breakage of the covalent backbone of a DNA molecule, and the term “cleavage domain” refers to a polypeptide sequences which possesses catalytic activity for DNA cleavage.

The cleavage domain can be obtained from any endo- or exonuclease. Exemplary endonucleases from which a cleavage domain can be derived include, but are not limited to, restriction endonucleases and homing endonucleases. These enzymes can be used as a source of cleavage domains. In addition, both single-stranded cleavage and double-stranded cleavage are possible, in which double-stranded cleavage can occur depending on the source of cleavage domains. In this regard, the cleavage domain having double-strand cleavage activity may be used as a cleavage half-domain. Herein, the cleavage domain can be used interchangeably for single-stranded cleavage and double-stranded cleavage.

The term “endonuclease” refers to any wild-type or variant enzyme or chemical endonuclease capable of catalyzing the hydrolysis (cleavage) of bonds between nucleic acids within of a DNA or RNA molecule, for instance a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer 2005). Such chemical endonucleases are comprised in the term “endonuclease” according to the present invention. In the scope of the present invention is also intended any fusion between molecules able to bind DNA specific sequences and agent/reagent/chemical able to cleave DNA or interfere with cellular proteins implicated in the DSB repair (Majumdar et al. 2008, Liu et al. 2009).

A cleavage domain can be derived from any nuclease or portion thereof. In general, two fusion proteins are required for cleavage if the fusion proteins comprise cleavage half-domains having double-strand cleavage activity. Two cleavage half-domains can be derived from the same endonuclease (or functional fragments thereof), or each cleavage half-domain can be derived from a different endonuclease (or functional fragments thereof). In addition, binding of two fusion proteins to their respective target sites places the cleavage half-domains in a spatial orientation to each other that allows the cleavage half-domains to form a functional cleavage domain, e.g., by dimerizing. Thus, any integral number of nucleotides or nucleotide pairs can intervene between two target sites (e.g., from 2 to 50 nucleotide pairs or more).

In one embodiment of the present disclosure the composition comprises an agent introducing double-strand breaks (DSB) within the genome. A double strand break agent may be selected from the group comprising site-specific nucleases, restriction endonucleases, homing endonucleases, meganucleases or chemical endonucleases.

In certain embodiments, the method of the present disclosure facilitates an increase in the frequency of homologous recombination (HR) or non-homologous end joining (NHEJ events) by lowering nucleosomal occupancy.

The terms “target sequence”, “target gene” or “target locus” as used herein, refer to a distinct chromosomal location, polynucleotide sequence or a gene in the chromosome selected for alteration by gene targeting. In other words, the nucleotide changes may be introduced into either a gene or a site that is not part of a genomic sequence. In certain cases, the target sequence/gene may contain a mutation that needs to be repaired or replaced. By “mutation” is intended the substitution, the deletion, and/or the addition of one or more nucleotides/amino acids in a nucleic acid/amino acid sequence. Alternatively, the target gene needs to be attenuated, inactivated, or replaced with a heterologous sequence/gene. To achieve high rate of gene targeting according to the present invention, a site of interest within workable proximity of the target sequence or within the target sequence may contain a DNA binding sequence recognizable by a cleavage agent, for example an enzyme that can make a double stranded break at or near this site.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) introducing into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining.

During gene targeting, it may be desired to manipulate a eukaryotic cell at multiple sites within the same genome. As used herein, the term “multiple” refers to at least one target site. It may also refer to two, three, four, five six or more target sites within the genome of the same eukaryotic cell that are being targeted. Herein, at least one or more double-strand break agent(s) with specificity for at least one or more target sites within the same genome are introduced into the cell. Furthermore, at least one or more targeting cassette having sufficient sequence identity with at least one or more target sites are introduced into the same cell.

Accordingly, in one embodiment, the present invention relates to a method wherein the frequency of insertion of a nucleotide sequence of interest in at least one or more specific chromosomal site(s) within the genome of a eukaryotic cell is increased comprising the steps of (i) lowering nucleosomal occupancy, (ii) cleaving the DNA at one or more pre-determined site(s) in the genome using site-specific nucleases and (iii) introducing into said eukaryotic cell a composition comprising one or more targeting cassette(s) comprising a polynucleotide of interest and at least a first region having sufficient sequence identity to a corresponding first region of a target site in said eukaryotic genome, wherein said polynucleotide of interest is inserted into at least one or more target site(s) by a homologous recombination event or non-homologous end joining.

As used herein, the term “targeting cassette” (also referred to as “targeting DNA construct” or “donor construct” or “repair matrix”) refers to a nucleic acid introduced in a cell for altering a target sequence in chromosomal DNA. A targeting cassette can be used for purposes such as modifying, replacing, attenuating, mutating or inactivating a target sequence. A targeting cassette may also be used to insert a large stretch of new sequence at a particular position. For example, in a process termed “transgenesis” a desired gene sequence (transgene) may be inserted at a position that is expected to provide expression of the gene at therapeutically effective levels or it may provide expression of other elements. Such elements may be selectable markers (e.g., a positive selectable marker such as an antibiotic resistance marker), promoter elements, non-selectable marker protein coding nucleic acid (e.g., nucleic acid encoding cytokines, growth factors, antibodies etc.). Inserts may also encode detectable proteins such as luciferase and fluorescent proteins such as green fluorescent protein and yellow fluorescent protein). A targeting cassette may include: (i) a polynucleotide sequence that is substantially identical to a region proximal to or flanking a target sequence and can be designated as the left and right arms of the targeting cassette; and (ii) a polynucleotide sequence which modifies the target sequence upon repair between the targeting cassette and the target sequence. Specifically, this polynucleotide sequence can be used to repair, modify, replace, attenuate or inactivate a target gene upon repair of the double-strand break between the targeting cassette and the target gene. The left and right arms of the targeting cassette refer to stretches of sequence which are homologous to flanking regions upstream and downstream of the DNA targeted. The targeting cassette can be part of a vector or not, linearized or not. Following cleavage of the DNA at the target site, a repair event is stimulated between the genome of the targeted cell at the site of cleavage and the targeting cassette, wherein the polynucleotide sequences between the flanking homologous sequences of the targeting cassette is inserted at the targeted genomic locus. Homologous sequences can have at least 50 bp, it can be more than 100 bp and or more than 200 bp are used.

In certain embodiments the site-specific nuclease is selected from the group comprising Transcription activator-like effector nucleases (TALEN), zinc-finger nucleases (ZFN), CRISPR/Cas9, CRISPR/nickase, megaendonucleases, NgAgo and chimeric nucleases.

The term “nuclease”, as used herein, refers to any polypeptide, or complex comprising a polypeptide, that can generate double stranded breaks in genomic DNA. Examples of nucleases include restriction endonucleases, chimeric nucleases and certain topoisomerases and recombinases.

As used herein, the term “transcription activator-like effector nuclease (TALEN)” refers to a class of highly specific restriction endonucleases that can be engineered to cut specific sequences of DNA and may include all known or commercial transcription activator-like effector nucleases. TALENs are fusion proteins comprising a TAL effector (TALE) DNA binding domain and a nucleotide cleavage domain. The TAL effector domain harbor highly conserved repeat domains that each bind to a single base pair of DNA. The identities of two residues (referred to as repeat variable di-residues or RVDs) in these 33 to 35 amino acid repeats are associated with the binding specificity of these domains. TAL effector repeats can be joined together to highly sequence specific restriction enzymes, which are capable of binding and cleaving target DNA sequences of interest.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be a transcription activator-like effector nuclease.

As used herein, the term “zinc finger nuclease” refers to a fusion protein comprising a zinc finger DNA-recognition and a nucleotide cleavage domain, and may include all known or commercial zinc finger nucleases.

As used herein, the term “zinc finger domain” refers to a protein that binds to a nucleotide in a sequence-specific manner through one or more zinc finger modules. The zinc finger domain includes at least two zinc finger modules. The zinc finger domain is often abbreviated as zinc finger protein or ZFP.

As used herein the term “zinc finger protein (ZFP)” refers to a polypeptide having nucleic acid (e.g., DNA) binding domains that are stabilized by zinc. The individual DNA binding domains are typically referred to as “fingers,” such that a zinc finger protein or polypeptide has at least one finger, more typically two fingers, or three fingers, or even four or five fingers, to at least six or more fingers. Each finger typically binds from two to four base pairs of DNA. Each finger usually comprises an about 30 amino acids zinc-chelating, DNA-binding region.

The nucleotide cleavage domain can be obtained from any endo- or exonuclease. Exemplary endonucleases from which a cleavage domain can be derived include, but are not limited to, restriction endonucleases. These enzymes can be used as a source of cleavage domains.

Restriction endonucleases are present in many species and are capable of sequence-specific binding to DNA (at a recognition site), and cleaving DNA at or near the site of binding. Certain restriction enzymes (e.g., Type IIs) cleave DNA at sites removed from the recognition site and have separable binding and cleavage domains. For example, the Type IIs enzyme FokI catalyzes double-stranded cleavage of DNA, at 9 nucleotides from its recognition site on one strand and 13 nucleotides from its recognition site on the other. Examples of the Type IIs restriction enzymes include FokI, AarI, AceIII, AciI, AloI, BaeI, Bbr7I, CdiI, CjePI, EciI, Esp3I, FinI, MboI, SapI, and SspD51, but are not limited thereto.

As used herein, the term “fusion protein” refers to a polypeptide formed by the joining of two or more different polypeptides through a peptide bond. The polypeptides may for example contain the zinc finger domain and nucleotide cleavage domain, which can cleave any target site in the nucleotide sequence. Methods for the design and construction of fusion proteins (or polynucleotide encoding fusion protein) may be any methods that are widely known in the art, and the polynucleotide may be inserted into a vector, and the vector may be introduced into a cell and the polynucleotide may contain a nuclear localization signal. A fusion protein may contain the TAL effector domain and nucleotide cleavage domain, which can cleave any target site in the nucleotide sequence.

Zinc finger nucleases may function as dimers, for example homodimers or heterodimers, to introduce DNA double strand breaks, thereby achieving the desired object of the present invention.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be a zinc finger nucleases.

As used herein, the terms “Clustered Regularly Interspaced Short Palindromic Repeats” and “CRISPR” refer to type II prokaryotic nucleic acid targeting proteins that were originally isolated from the bacterium Streptococcus pyogenes.

The CRISPR/Cas or the CRISPR-Cas system (both terms are used interchangeably throughout this application) does not require the generation of customized proteins (as in the case of technologies involving zinc finger proteins, meganucleases or transcription activator like effectors (TALEs)) to target specific sequences but rather a single Cas enzyme can be programmed by a short RNA molecule to recognize a specific DNA target, in other words the Cas enzyme can be recruited to a specific DNA target using said short RNA molecule.

In the bacterium Streptococcus pyogenes, four genes (Cas9, Cas1, Cas2, and Csnl) and two non-coding small RNAs (pre-crRNA and tracrRNA) act in concert to specifically bind to and degrade a target DNA. The specificity of binding to target nucleic acid is controlled by non-repetitive spacer elements in the pre-crRNA that, in conjunction with the tracrRNA, directs the Cas9 nuclease to a protospacer:crRNA heteroduplex and induces the formation of a double-strand break (DSB).

In general, “CRISPR system” or the “CRISPR-Cas system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex.

In aspects of the invention the terms “chimeric RNA”, “chimeric guide RNA”, “guide RNA”, “single guide RNA”, “synthetic guide RNA”, “sgRNA” and “gRNA” are used interchangeably and refer to the polynucleotide sequence comprising the guide sequence, the tracr sequence and the tracr mate sequence. The term “guide sequence” refers to the about 20 nt sequence within the guide RNA that specifies the target site and may be used interchangeably with the terms “guide” or “spacer”. The term “tracr mate sequence” may also be used interchangeably with the term “direct repeat(s)”.

As used herein, the term “CRISPR protein” refers to a protein comprising a nucleic acid (e.g., RNA) binding domain nucleic acid and an effector domain (e.g., Cas9, such as Streptococcus pyogenes Cas9). The nucleic acid binding domains interact with a first nucleic acid molecule either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise endonuclease domains, additional DNA binding domains, helicase domains, protein-protein interaction domains, dimerization domains, as well as other domains.

The term “CRISPR endonuclease” refers to an endonuclease (e.g., the Cas9 endonuclease) which, in combination with an RNA guide strand, can cause a double-strand break at a target the site in the genome.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be CRISPR/Cas.

The large Cas9 protein (>1200 amino acids) contains two predicted nuclease domains, namely HNH (McrA-like) nuclease domain that is located in the middle of the protein and a splitted RuvC-like nuclease domain (RNase H fold) (Haft et al. 2005, Makarova et al. 2006). The HNH nuclease domain and the Ruv-C domain have been found to be essential for double strand cleavage activity. Mutations introduced in these domains have respectively led to Cas9 proteins displaying nickase-activity, whereby the enzyme cleaves a single strand instead of having double-strand cleavage activity. Different inactivating mutation(s) of the catalytic residues in the RuvC-like domains produces a nickase able to cut one strand in position+3 bp (versus the 3′ end) respect with the Protospacer adjacent motif (PAM) location, a 2-6 base pair DNA sequence immediately following the DNA sequence targeted by the Cas9 nuclease. The mutation of the catalytic residue of the HNH domain generates a nickase able to cut the other strand in position+3 bp (versus the 5′ end) (Jinek et al. 2012). Introducing mutations in Cas9 resulting in nickase activity, instead of cleavage activity, can be used to produce cleavage at a given DNA target and increase the specificity in the same time. The method is based on the simultaneous use of nickase architecture of Cas9 (RuvC domain and/or HNH domain) and sgRNA(s) harboring two different complementary sequence to specific targets lowering the risk of producing off-site cleavage. By using at least one guide RNA harboring two different complementary sequence to specific targets or a combination of at least two guide RNA, the requirement for specificity passes from 12 to 24 nucleotides and, in turn, the probability to find two alternative binding sites of Cas9 (different from the ones coded in the two sgRNA) at an efficient distance from each other to produce an off-site cleavage becomes very low.

Accordingly, as used herein, the term “CRISPR/nickase” comprises a CRISPR complex with a Cas protein having one or more mutations and a first guide sequence directing cleavage of one strand of the DNA duplex near the first target sequence and a second guide sequence directing cleavage of the other strand near the second target sequence inducing a double strand break.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be CRISPR/nickase.

As used herein, the terms “homing endonuclease (HE)” and “meganuclease” refer to a class of double-stranded endonucleases having a large polynucleotide recognition site, at least 12 bp, for instance from 12 bp to 60 bp. A meganuclease is also called rare-cutting or very rare-cutting endonuclease and present a very low frequency of cleavage as they are long enough to occur only once in a genome and randomly with a very low probability (e.g., once every 7×10⁹ bp) (Jasin 1996). It includes any natural meganuclease such as a homing endonuclease, but also any artificial or man-made meganuclease endowed with such high specificity, either derived from homing endonucleases of group I introns and inteins, or other proteins such as Zinc-Finger proteins or group II intron proteins, or compounds such as nucleic acid fused with chemical compounds.

Meganucleases show high specificity to their DNA target, these proteins being able to cleave a unique chromosomal sequence and therefore do not affect global genome integrity. Natural meganucleases are essentially represented by homing endonucleases, a widespread class of proteins found in eukaryotes, bacteria and archaea (Chevalier and Stoddard 2001).

There are five different families of homing endonucleases. The members of the LAGLIDADG family of homing endonucleases each have one or two LAGLIDADG motifs per polypeptide chain. The LAGLIDADG amino acid sequence is a conserved sequence directly involved in domain-domain and subunit-subunit interaction and the DNA cleavage process. Those enzymes that have only one motif per polypeptide chain act as homodimers, while those having two motifs act as monomers. The members of the GIY-YIG family of homing endonucleases have one GIY-YIG motif as the catalytic motif that is associated with a DNA binding motif. The prototypic enzyme of this family is 1-TevI. The members of the His-Cys box family of homing endonucleases contain a stretch of 30 amino acids including two conserved histidines and three conserved cysteins. I-Ppol is a member of said family and acts as a monomer. The members of the H—N—H family of homing endonucleases are characterized by a consensus sequence of approximately 30 amino acids having two pairs of conserved histidines and one asparagine. The said conserved amino acids form an alpha-beta-beta-alpha (αββα) metal finger motif. The PD . . . D/EXK family of homing endonucleases are characterized by a structural core that consists of a four-stranded beta sheet flanked by alpha helices that harbors the characteristic PD . . . D/EXK active site motif (see, e.g., Pingoud et al. 2005).

In particular, artificial meganucleases include the so-called “custom-made meganuclease” which is a meganuclease derived from any initial meganuclease, either natural or not, presenting a recognition and cleavage site different from the site of the initial one. By “different” is intended that the custom-made meganuclease cleaves the novel site with an efficacy at least 10 fold more than the natural meganuclease, for instance at least 50 fold, for instance at least 100 fold. “Natural” refers to the fact that an object can be found in nature. For example, a meganuclease that is present in an organism, that can be isolated from a source in nature and which has not been intentionally modified by man in the laboratory is natural.

The custom-made meganuclease may be prepared by the targeted mutagenesis of the initial meganuclease. The diversity is introduced at positions of the residues contacting the DNA target or interacting (directly or indirectly) with the DNA target. The diversity is for instance introduced in regions interacting with the DNA target, for instance introduced at the positions of the DNA-interacting amino acids within the nucleic acid binding domain.

Functional HE variants having specificity for a target sequence of interest can be identified and isolated by the methodology described in mutations of the natural recognition site. The specificity of existing HEs may be modified by introducing a small number of variations to the amino acid sequence.

An alternative approach for generating target sequence specific HEs involves exploiting HEs' high degree of natural diversity via fusing domains from different molecules. This approach makes it possible to develop chimeric HEs with new recognition sites that are composed of a half-site of a first HE and a half-site of a second HE. By, for example, fusing the protein domains of I-Dmol and I-CreI, the chimeric HEs E-DreI and DmoCre were created (Chevalier et al. 2002). These HEs can be further combined to generate functional chimeric HEs having a desired target sequence specificity and can, therefore, be adapted for use in the fusion proteins of the present disclosure.

Within certain aspects, the fusion proteins disclosed herein may comprise a target specific homing endonuclease variant such, for example, a target specific variant of a homing endonuclease selected from the group consisting of I-HjeMI, I-CpaMI, I-OnuI, I-CreI, PI-SceI, I-SceII, I-Dmol, I-TevI, I-TevII, I-TevIII, I-Ppol, I-PpoII, I-HmuI, I-HmuI, I-SSp68031, I-AniI, I-CeuI, I-ChuI, I-CpaI, I-CpaII, H-DreI, I-LlaI, I-MosI, PI-PfuI, PI-PkoII, I-PorI, PI-PspI, I-ScaI, I-SecIII, I-SceIV, I-SceV, I-SceVI, I-SceVII, PI-TLiI, PI-TLilI, I-Tsp061I, and I-Vdi141I.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be homing endonuclease or meganuclease.

As used herein, the term “Natronobacterium gregoryi Argonaute (NgAgo)” refers to a DNA-guided DNA endonuclease from the family of Argonaute proteins and was first identified in the bacterium Natronobacterium gregoryi.

Argonautes are a family of endonucleases that use 5′ phosphorylated short (13-25 nt) single-stranded nucleic acids as guides to cleave targets (Gao et al. 2016). The argonaute protein from the bacterium Natronobacterium gregoryi can cleave genomic DNA in mammalian cells, wherein 5′ phosphorylated ssDNA is loaded onto the Argonaute complex, which guides the endonuclease to DNA targets and catalyzes cleavage of the DNA.

NgAgo uses DNA-DNA hybridization to target cleavage sites with high accuracy and enables the cleavage of genomic sequence by a single protein.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be NgAgo.

As used herein, the term “chimeric nucleases” refers to a chimeric protein that is designed to create a double-stranded break at one or more selected sites in the chromosome. Chimeric nucleases comprise one or more specific DNA binding domains and one or more cleavage domains. The DNA binding domains confer the DNA binding specificity, while the cleavage domains confer the double-stranded break activity. A chimeric nuclease can be made as a fusion protein or by linking the DNA binding domain(s) to the cleavage domain(s).

A variety of DNA binding domains are known in the art, and any DNA binding domain that recognizes the desired site with sufficient specificity may be employed. DNA binding domains include zinc finger binding domains.

Cleavage domains may derive from any nuclease that has DNA cleavage activity. Examples of protein types having cleavage domains include restriction enzymes, topoisomerases, recombinases, integrases and DNases. Construction of a chimeric nuclease is generally simplified if the cleavage domain is obtained from a nuclease that has separate domains for sequence recognition and DNA cleavage.

Restriction endonucleases are present in many species and are capable of sequence-specific binding to DNA (at a recognition site), and cleaving DNA at or near the site of binding. Certain restriction enzymes (e.g., Type IIS) cleave DNA at sites removed from the recognition site and have separate sequence recognition domains and cleavage domains. For example, the Type IIS restriction enzyme FokI catalyzes double-stranded cleavage of DNA, at 9 nucleotides from its recognition site on one strand and 13 nucleotides from its recognition site on the other. Other examples of Type IIS restriction enzymes include AarI, AceIII, AciI, AloI, BaeI, Bbr7I, CdiI, CjePI, EciI, Esp3I, FinI, MboI, SapI, or SspD51, but are not limited thereto, more specifically, see Roberts et al. 2003.

Chimeric nucleases can form dimers (e.g., via binding to two cognate DNA binding sites within a target sequence). For example, chimeric nucleases can form a homodimer between two identical chimeric nucleases (e.g., via binding to two identical DNA binding sites within a target sequence). Alternatively, chimeric nucleases can form a heterodimer between two different chimeric nucleases (e.g., via binding to two different DNA binding sites within a target sequence). Methods of making chimeric nucleases are described in the art.

Accordingly, in one embodiment, the present invention relates to a method wherein the frequency of insertion of a nucleotide sequence of interest in a specific chromosomal site within the genome of a eukaryotic cell is increased comprising the steps of (i) lowering nucleosomal occupancy, (ii) cleaving the DNA at a pre-determined site in the genome using site-specific nucleases and (iii) into said eukaryotic cell a composition comprising a targeting cassette comprising a polynucleotide of interest and at least a first region having sufficient sequence identity to a corresponding first region of a target site in said eukaryotic genome, wherein said polynucleotide of interest is inserted into the target site by a homologous recombination event or non-homologous end joining. The site-specific nuclease may be a chimeric nuclease.

An ideal genome editing reagent would only create a double strand break point at one or more “on-target” sites in the genome. However, current genome editing reagents often generate double-stranded break points at one or more “off-target” sites in the genome.

Accordingly, in one embodiment of the present disclosure the frequency of insertion of a nucleotide of interest at an unintended chromosomal site within the genome is reduced.

Accordingly, in one embodiment of the present disclosure the frequency of insertion of a nucleotide sequence of interest in a specific chromosomal site within the genome of a eukaryotic cell is increased and the frequency of insertion of a nucleotide of interest at an unintended chromosomal site within the genome is reduced.

In one embodiment, the subject methods may be used to introduce a transgene for expression in the cell. For example, a genetic disease caused by a decrease in the level of a necessary gene product may be treated or ameliorated by providing a transgene expressing the needed gene product. The invention further provides a method for producing at least one recombinant protein in a cell by providing a transgene expressing the desired recombinant protein. The transgene may be targeted to the location of the endogenous gene, or to a different location. Such methods may comprise: (i) introducing an HMGB1 modulator into a cell, (ii) introducing a DSB agent into a cells and (iii) introducing a targeting cassette into the cell under conditions appropriate for introducing the targeting cassette into the site of interest, wherein said targeting cassette comprises: (i) a nucleic acid sequence that is substantially identical to one or more regions proximal to or flanking a target sequence in chromosomal DNA; and (ii) a nucleic acid sequence which replaces the target sequence upon recombination between the targeting cassette and the target sequence.

As used herein, the term “transgene” refers to a sequence encoding a polypeptide intended to be introduced into a cell, tissue or organism by recombinant technologies. The polypeptide encoded by the transgene may be either not expressed, or expressed but not biologically active, in the cell, tissue or organism in which the transgene is inserted.

In another embodiment, the present invention provides methods for ameliorating, treating or preventing a disease in an individual, wherein the disease is caused in part or in whole by a genomic target sequence. Such methods may comprise: (i) introducing an HMGB1 modulator into a cell, (ii) introducing a DSB agent into a cells and (iii) introducing a targeting cassette into the cell under conditions appropriate for introducing the targeting cassette into the site of interest, wherein said targeting cassette comprises: (i) a nucleic acid sequence that is substantially identical to one or more regions proximal to or flanking a target sequence in chromosomal DNA; and (ii) a nucleic acid sequence which replaces the target sequence upon recombination between the targeting cassette and the target sequence, whereby the genetic disease is ameliorated, treated or prevented. The individual may be a human.

There are now described kits, which contain components useful for conveniently practicing the methods described. In certain embodiments, the In one embodiment, such a kit contains a double-strand break agent, a chromatin modifier and a targeting cassette. The chromatin modifier may be a modulator of HMGB1. For instance, HMGB1 can lower nucleosome occupancy. The HMGB1 modulator may be selected from the group comprising Glycyrrhizin or derivatives thereof, Tanshinone IIA or derivatives thereof, Epigallocatechin-3-gallate (EGCG), Quercetin, Lycopene, nafamostat mesilate, gabexate mesilate, ethyl pyruvate, carbenoxolone or Oligonucleotide (ODN)-based inhibitors of HMGB1. A kit may also comprise a double-strand break agent. The double-strand break agent may be selected from the group comprising Transcription activator-like effector nucleases (TALEN), zinc-finger nucleases (ZFN), CRISPR/Cas9, CRISPR/nickase, megaendonucleases, NgAgo and chimeric nucleases. Optionally, the double-strand break agent may be delivered as a vector or vector system, wherein the double-strand break agent is delivered into the cell in a nucleotide construct that encodes and expresses the double-strand break agent. Optionally, the nuclease may be under the control of an inducible promoter. Alternatively, the double-strand break agent may be delivered to the cell as a protein or as a ribonucleoprotein or as an mRNA. Different delivery methods are known that can be employed by those of skill in the art to deliver the double-strand break agent to the nucleus of the cell.

As used herein, the terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non-chromosomal, semi-synthetic or synthetic nucleic acids. It can be delivered to the cell as naked nucleic acid, as a complex with one or more delivery agents (e.g., liposomes, poloxamers) or contained in a viral delivery vehicle, such as, for example, an adenovirus or an adeno-associated Virus (AAV). Vectors may be capable of autonomous replication (episomal vector) and/or expression of nucleic acids to which they are linked (expression vectors). The vectors of the system may further comprise one or more nuclear localization signal(s) (NLS). Large numbers of suitable vectors are known to those of skill in the art and commercially available.

Also provided are kits that comprise of (i) at least one (e.g., two, three, four, or five) HMGB1 modulators (e.g., any of the HMGB modulators described herein); (ii) a targeting cassette and (iii) at least one (e.g., two, three, four, or five) double-strand break agents (e.g. any of the DSB agents described herein). In some embodiments, the kit includes only one HMGB1 modulator. In other examples, the kit includes at least two (e.g., two, three, four, five, six, seven, eight, nine, or ten) HMGB1 modulators. In some embodiments, the kit includes only one DSB agent. In other examples, the kit includes at least two (e.g., two, three, four, five, six, seven, eight, nine, or ten) DSB agents. In some embodiments, only one chromosomal site is targeted. In some embodiments, at least two (e.g., two, three, four, five, six, seven, eight, nine, or ten) chromosomal sites are targeted.

A kit may comprise detailed instructions explaining how to perform gene targeting.

All patents, published patent applications, publications, references and other material referred to herein are incorporated by reference herein in their entirety.

As used herein, the term “comprising” encompasses “including” as well as “consisting,” e.g. a composition “comprising” X may consist exclusively of X or may include something additional, e.g., X+Y.

As used herein, the term “about” in relation to a numerical value x means, for example, +/−10%. As used herein, the word “substantially” does not exclude “completely,” e.g. a composition which is “substantially free” from Y may be completely free from Y. Where necessary, the word “substantially” may be omitted from the definition of the disclosure.

As used herein, the term “patient” and “subject” includes any human or nonhuman animal and can be used interchangeably. The term “nonhuman animal” includes all vertebrates, e.g. mammals and non-mammals, such as nonhuman primates, sheep, dogs, cats, horses, cows, camels, chickens, amphibians, reptiles, etc.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-ld) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

In addition, in the context of the present invention, the term “antibody” shall also encompass alternative molecules having the same function of specifically recognizing proteins, e.g. aptamers and/or CDRs grafted onto alternative peptidic or non-peptidic frames.

As used herein, the term “nucleic acid” refers to polynucleotides such as deoxyribonucleic acid (DNA), and, where appropriate, ribonucleic acid (RNA). The term should also be understood to include, as applicable to the embodiment being described, single-stranded (such as sense or antisense) and double-stranded polynucleotides. This term includes both naturally occurring nucleotide and artificially modified nucleotides.

It will be understood that unless indicated to the contrary, terms intended to be “open” (e.g., the term “including” should be interpreted as “including but not limited to,” the term “having” should be interpreted as “having at least,” the term “includes” should be interpreted as “includes but is not limited to,” etc.). Phrases such as “at least one,” and “one or more,” and terms such as “a” or “an” include both the singular and the plural.

Without intending to limit the scope of the disclosure in any way, it is further described by way of illustration of the following example.

### EXAMPLES

Materials and Methods:

Yeast Growth, Cell Cycle Arrests and Flow Cytometry:

Unless otherwise stated, yeast cultures were grown at 30° C. until logarithmic (LOG) growth-phase (OD600=0.7; 1×10⁷ cells/ml) prior to Zeocin (Invitrogen) or yIR exposure at 30° C. Live cell microscopy was done at 25° C. Flow cytometry samples were prepared as previously described (Haase and Lew 1997).

For controlled GAL1-10::H3/H4 expression experiments coupled with gene targeting assays, GA-8386 and the relevant control strain cultures (GA-8385) were grown overnight to saturation in YP galactose/raffinose (YP Gal/Raff 1:5) medium. The next morning, cultures were inoculated in the same respective medium and grown until logarithmic (LOG) growth-phase (OD600=0.7; 1×10⁷ cells/ml) prior to pulsed histone level reductions. After reaching LOG phase, cells were washed once and pulsed histone H3 and H4 level reductions were accomplished via grown in either pre-warmed 30° C. YP galactose/raffinose 1:20 or YP raffinose medium for 120 minutes prior to transformation with the respective gene targeting selection cassettes.

For cell cycle arrest and release experiments, 1.5×10⁻⁸ M alpha factor (Zymo Research) was added to exponentially growing cultures at a density of OD600=0.5. After 1 hour, another half of the initial alpha factor amount was added for 30 minutes and cells were either held in G1 phase or released into pre-warmed medium for 15-25 minutes prior to Zeocin damage treatment in S phase. Cell fixation in the relevant experiments was done for 2 minutes at room temperature with 4% Paraformaldehyde.

For all Zeocin or γIR exposure experiments, saturated yeast overnight cultures were diluted to OD600=0.1 the next morning and grown to LOG phase. In all assays, Zeocin was added directly to G1 arrested, S phase released or asynchronously growing LOG cultures. Cultures were incubated with the drug for 1 h prior to high-speed tracking microscopy or the indicated amount time periods for other assays and experiments. For γIR exposure, 5 ml of cell culture was transferred to a 35×10 mm petri dish and irradiated in a Faxitron CellRad cell-irradiator until the indicated dose (Grey) was reached. After γIR treatment, cells were directly harvested for further downstream Western blot or mass-spectrometry-based analysis. For undamaged conditions, cells were either imaged immediately for high-speed tracking microscopy or growth was continued along with the treated samples for the indicated time periods. γIR undamaged control cells were also spread on petri dishes and harvested after irradiation of treated cells was completed. Further specific growth and treatment conditions for high-speed tracking live cell microscopy were done according to Seeber et al (Seeber et al. 2013).

For H3-CFP (Strain GA-3364 and derivatives) and 2-foci (Strain GA-9777) live cell fluorescent microscopy, LOG phase cells were trapped with 3 pulses of 5 psi pressure in CellASIC plates of the ONIX microfluidic perfusion system (Merck Millipore). All perfusions were done at a continuous flow rate of 2 psi pressure. After a 20-30 minute recovery phase, cells were treated for 30 minutes with the indicated amount of Zeocin prior to high-speed CFP-RFP tracking microscopy. The recovery phase of H3-CFP tagged cells was 20 minutes after which they were treated with a pulse of Zeocin for 1 hour and H3-CFP fluorescence was followed for additional 40 minutes after treatment.

Genome-Wide Nucleosome Mapping:

Strains tested for changes in nucleosome occupancy (GA-6879 and GA-8386) were grown in appropriate media to OD600=0.8. Cultures were split into two and one of them was treated with Zeocin (500 μg/ml) for 1 hour. At this point the OD600 absorbance of each sample was measured and Candida glabrata cells were spiked in to 1/10 according to the sample OD600. Cells were washed three times with ice cold TBS (20 MM Tris.HCl pH8.0 and 150 mM NaCl) and lysed by beat beating in MNase digestion buffer (10 mM Tris pH 8.0, 50 mM NaCl, 5 mM MgCl2, 1 mM CaCl₂), 1 mM beta-mercaptoethanol, 0.5 mM spermidine, 0.075% NP40). The obtained chromatin samples were MNase digested to isolate mono-nucleosomes and sequencing libraries were prepared according to the method described in Wiechens, N. et al. (Wiechens et al. 2016). Paired end libraries of MNase digested chromatin were sequenced using Illumina HiSeq technology. Fastq files containing raw reads were aligned to the S. cerevisiae and C. glabrata reference genomes by Bowtie2 with option of maximum fragment length 500 for nucleosome fragments. The nucleosome dyads at each position were calculated in a defined window flanking the transcription start site (TSS). The sum of dyads at a given position across all TSS was then normalized by the total number of nucleosome dyads across all position flanking ˜6000 TSSs in the given window. The reads were further normalized by dividing the fraction of C. glabrata reads in the sample. For low and high expression gene plots, the TSS of 15% highly and 15% lowly expressed genes were chosen. The data was smoothed using a 50 bp sliding window for graphical representation. Plots were generated with python's plotting modules matplotlib and pylab.

Quantitative Western Blot Analysis:

The total protein content in the relevant samples was determined with the Quant-iT protein assay kit (Thermo Scientific) and 8.75 μg of total protein was loaded and run on Criterion TGX Stain-Free 8-16% (Biorad) gels under SDS denaturing electrophoresis conditions. Rapid fluorescent detection of all proteins in the gel or on the membrane was done according to the manufacturer's specifications and protein transfer on PVDF membranes was performed using the Trans-Blot Turbo system. Rad53 protein was detected using a custom-made mouse monoclonal antibody (GenScript) against FHA2 domain of Rad53. Anti-yH2A was similarly a custom-made polyclonal antibody, that is specific for phospho-S129 in yeast H2A.

Chromatin Fractionation and Quantitative Mass Spectrometry:

For SILAC based mass spectrometry, lysine and arginine double labeling of the lys2A arg4A strain yAG-06A was achieved by growth for at least ten generations in “heavy” medium as described previously in Gruhler et al. 34. After growth to LOG phase or at G1 cell cycle arrest, “light” labeled cells (or “heavy” labeled cells for label-swap controls) were treated for 1 h with Zeocin and mixed 1:1 based on exact cell count with “heavy” labeled (“light” for label-swap control), non-treated control cells. Prior to mixing, FACS and Western blot samples were taken to test for cell cycle distribution and DDC activation.

Chromatin fractionation was performed as previously described with the modification that chromatin obtained from SILAC labeled yeast samples was resuspended in Urea Buffer (50 mM TRIS pH 7.5, 6 M Urea, 1% SDS, 5 mM EDTA) sonicated for optimal solubilizing of proteins followed by a TCA protein precipitation step prior to downstream mass spectrometric analysis. To avoid carbamylation in urea buffer, samples were kept below 20° C. and quickly processed. Control samples from whole cell extract (WCE), supernatant (SUP) and chromatin fraction (CHR) were analyzed with SDS-PAGE (Novex 8-16% Tris-Glycine Gel, Invitrogen) gel electrophoresis followed by Coomassie staining.

Samples for label-free histone quantification came from LOG phase or G1 phase arrested cells grown in YPD medium. After gammaIR treatment, 5 ml of culture were fixed with 10% TCA on ice. Whole cell lysates were obtained with bead-beating cells at 4° C. in urea buffer (50 mM TRIS pH 7.5, 6 M Urea, 1% SDS, 5 mM EDTA). 100-150 μg total protein was precipitated for downstream MS analysis.

For both SILAC and label-free samples were reduction and alkylation of cysteines was performed by adding 45 mM DTT for 30 min followed by 100 mM iodoacetamide for another 30 min (in the dark), both at room temperature. Prior to the addition of 20 μl of 1 mg/ml LysC (Wako, Japan) the extracts were twofold diluted to keep a final HEPES concentration of 20 mM. The first digest was performed overnight at 25° C. After 2-fold dilution, 100 μl of 0.5 mg/ml trypsin was added and the second digest was performed at 37° C. overnight. Samples were desalted using SepPak C18 columns (Waters) and eluates were dried to completion in a SpeedVac (Thermo Scientific).

Both SILAC and label-free LC/MS/MS analyses was performed on an Easy-nLC 1000 pump coupled to an LTQ Orbitrap Velos mass spectrometer (Thermo Scientific) using a Digital PicoView ion source (New Objective). Peptides were separated on a New Objective analytical column (75 μm×25 cm, Reprosil, 3 μm) with a 165 minute acetonitrile gradient. The flow rate was 200 nL/min and injection volumes were adapted accordingly for 1 μg peptides on column. Data were acquired in a Top20 data dependent analysis mode. MS scans were acquired at a resolution of 60,000 over a range of m/z 350 to 1200. Peptides were identified searching SwissProt using Mascot Distiller and Mascot (Matrix Science) considering acetylation at protein N-terms, deamidation at asparagine and glutamine, oxidation at methionine and phosphorylation at serine as well as at threonine. Two missed cleavage sites were allowed. Results of label-free samples were compiled in Scaffold 3.0 (Proteome Software), SILAC quantification and results were obtained with the MaxQuant software.

Label-free relative quantification of histones was done by generating the extracted ion chromatogram for the peptide precursor mass, integrating the peak areas which are then used for calculating the peptide ratios. The average of those ratios determines the ratio of the histones (reference untreated or wild type sample). This method is more precise than the TOP 3 TIC method used in Scaffold. Untreated or wild type references were set to 1. We used 2 peptides from each ALF, KPK1, IF4A and IFSA1 protein as internal references for the quantification of relative histone abundances in each run. Histone level ratios in SILAC samples are shown as the average from all non-label-swap or label-swap replicas. Ratios were derived from the MaxQuant peptide list taking in account only core histone peptides reported as not being subject to post translational modifications2l. Significance was addressed by blotting the distribution of all protein ratios from the MaxQuant protein-groups list together with the protein intensities. Core histones were always the most abundant proteins measured and reside within the first significant interval. The MaxQuant protein-groups list was filtered by removing all contaminants, all reverse hits and proteins quantified with less than 2 peptides. The cutoff for variability was set to 30%. Normalization was done manually taking the 35 most abundant proteins (histones excluded). The MaxQuant peptide list was filtered accordingly without variability cutoff and only taking peptides into account that had a L/H or H/L count greater than 3. Normalization was done manually taking the top 10% most abundant peptides (histone peptides excluded).

Live Cell Microscopy and Image Analysis:

Live microscopy was done on a temperature controllable Olympus IX81 microscope with a Yokogawa CSU-X1 scanning head equipped with two EM-CCD EvolveDelta (Photometrics) cameras, an ASI MS-2000 Z-piezo stage and a PlanApo x100, NA 1.45 total internal reflection fluorescence microscope oil objective and Visiview software. For mRFP-GFP or mRFP-CFP high-speed tracking, fluorophores were excited with lasers at 561 nm (mCherry or mRFP) and 491 nm (GFP) or 440 nm (CFP) and emitted fluorescence was acquired simultaneously on separate cameras (Semrock FF01-617/73-25 filter for mCherry/mRFP and Semrock FF02-525/40-25 filter for GFP or Semrock FF01-475/42-25 for CFP). High-speed time-lapse series were conducted taking 8 optical slices per stack either every 80 ms for 1 min or 300 ms for 2 min, with 10 ms exposure times per slice respectively. Time-lapse image stacks were analyzed as in Dion et al. 9, using a custom made ImageJ (FIJI) plug-in36 to extract coordinates of locus position from the movies. Phototoxicity was tested by exposing wild-type cells (GA-6879) to standard imaging conditions and following outgrowth for 5 h by morphological analysis, comparing them with unexposed cells. Time-series acquired from Strains GA-9227 and GA-9777 (Two-spot data) were deconvolved using Huygens Remote Manager, channel-aligned and cropped to contain one single cell/nucleus with the two respective fluorescent spots. Spot tracking over time was done with the ImageJ plugin TrackMate included in Fiji37. Boxplot graphs were generated by plotting all measured distances of treated or untreated cells. Relative MSD analysis was performed with KNIME38. For each frame, the distance vector of tracks in two channels was measured by selecting the two spots with minimal distance. The inventors performed an MSD analysis on the distance vectors for all frames and tracks with a maximum MSD(t) value bigger than 10 μm² were considered as outliers (due to mis-matching two distant tracks) and removed from the analysis. Relative MSD vs. t was averaged over all tracks and plotted using R.

Structured Illumination Microscopy and Image Analysis:

Structured illumination images were acquired on a Zeiss Elyra S.1 microscope with a Andor iXon 885 EMCCD camera using a HR diode 488 100 nW solid state laser, BP 525-580+LP 750 filter and a PLAN-APOCHROMAT 63×N.A. 1.4 oil DIC objective lens. Cells were first fixed in PFA 4%, washed 3 times in PBS and then attached to a thin SIM grade Zeiss 1.5 glass coverslip using Concanavalin A. Cells were fully sectioned by 50-65 slices with 0.1 nm intervals taken at 60 ms exposures per slice using 5 rotations of the illumination grid. Brightfield images of the cells were also acquired using an X-Cite PC 120 EXFO Metal Halide lamp. Zen Black was used to process the images using automatic settings with the Raw Scale option selected. 3D stacks were then analyzed by using pixel classification and a custom Matlab script to determine the spot volumes and other features as follows. We used a fully automated nucleus and spot segmentation workflow that allowed the individual detection and feature extraction where a manual or even a semi-automated delineation would be unfeasible. The image processing software was realized within the MATLAB environment and supported by the supervised learning-based pixel classification toolkit Ilastik39. The voxels corresponding to the nucleus, the inner spot and background regions are annotated interactively by brush strokes during the training phase. Features calculated at the labeled pixels and their local neighborhood are then used to train a pixel classifier based on a Random Forest ensemble learning method. The processing software provides an automated whole segmentation of all the nuclei and spots present in the scene. The image processing function is later used in a parallelized batch process on multiple processors. After detection and segmentation of nuclei and spots, the program produces a graphical output in form a maximum intensity projection with delineation of the nucleus, the spots and the unique ID integer that identifies the nucleus candidate. In addition, 3D logical masks corresponding to the classes “spot” and “nucleus” are computed. Finally, the program generates an ascii file where the key features like volume and solidity 3D and descriptive statistics are listed for all detected nuclei and foci. The solidity factor is calculated as the proportion of pixels in the 3D convex hull. For statistical analysis and data representation, raw volumes were filtered to exclude spots smaller than 200 and greater than 4000 voxels, the control (Ctr.) condition was set to 1 and Zeocin treated spot or nuclei volume distributions are shown relative to the untreated control. The distributions were plotted with R as boxplot graphs or a cumulative density functions.

Ectopic Recombination Assays:

As used in WT cells, cells depleted for NHP6A/NHP6B and in arp8Δcells. For specific growth conditions, please consult the “Yeast growth, cell cycle arrests and flow cytometry” section. Equal amounts of exponentially growing WT (GA-6879) arp8Δ(GA-8132) and nhp6Δ(GA-9771) were transformed with the transformation protocol either with a linearized URA3 plasmid (pRS406 cut with StuI) presenting 800 bp homology to the W303 ura3-1 locus or a mgs1::caURA3 PCR fragment (template plasmid #1050) presenting 40 bp and 42 bp upstream and downstream homology to the MGS1 locus. As a control, the centromeric circular plasmid #2422 (ADE2, hphMX4, Cen/ARS), which is maintained in yeast cells ectopically, was transformed alongside with the URA3 integration cassettes. After transformation, cells were split and plated on SC-URA plates (100 μL) to select for transformants resulting from integration and on and YPD+Hygromycin B plates to select for cells containing the plasmid. The numbers of Ura+ and Leu+transformants obtained from each reaction were compared to calculate the relative integration rate for each strain, with that of a wild-type strain arbitrarily set to 1 as a reference. Growth was scored in biological quadruplicates and each transformation was done with four technical replicates; results were averaged.

As used in “Ctr.” cells and Gal:H3/H4 “histone shutdown” cells. After pre-growth in YP Galactose/Raffinose 1:5 medium, equal amounts of exponentially growing control (GA-8385) and Gal:H3/H4 “histone shutdown” (GA-8386) cells were pulse-reduced for histone H3 and H4 levels via 2 hour growth in either YP Galactose/Raffinose 1:20 or YP Raffinose medium. After the histone-reduction pulse, transformations were done with either an atg2::hphMX4 PCR fragment (PCR product—ATG2::hygro, template plasmid #1049) presenting 40 bp and 40 bp upstream and downstream homology to the ATG2 locus or a mgs1::hphMX4 PCR fragment (PCR product—MGS1::hygro, template plasmid #1049) presenting 40 bp and 42 bp upstream and downstream homology to the MGS1 locus. As a control, the centromeric circular plasmid #282 (LEU2, Cen/ARS), which is maintained in yeast cells ectopically, was transformed alongside with the hphMX4 PCR integration cassettes.

After transformation, cells were split and plated on YPGal+Hygromycin B plates (100 μL plated) to select for transformants resulting from integration of ATG2::hygro or MGS1::hygro and on and SCGal-LEU plates (10 μL plated) to select for cells containing the plasmid. The numbers of hphMX4+ and LEU+transformants obtained from each reaction were compared to calculate the relative integration rate for each strain, with that of a wild-type strain arbitrarily set to 1 as a reference. Growth was done in biological quadruplicates and each transformation was done with four technical replicates; results were averaged.

Results

DNA Damage Triggers Extensive Histone Loss from Chromatin:

To investigate whether DNA damage and DDC activation affect chromatin structure and/or composition genome-wide, the inventors used quantitative SILAC mass spectrometry in Saccharomyces cerevisiae and measured histone abundance before and after acute treatment (1 hour) with the radiomimetic drug Zeocin. Relative ratios of non-modified histone peptides (damage over control—L/H) indicate a substantial loss of 20-40% of all core histones H2A, H2B, H3 and H4. Interestingly, levels of the histone variant Htz1 (H2A.Z) remained stable. Quantitative immunoblot analysis confirmed our observations and showed robust DDC activation (yH2A signal) along with a dose-dependent relationship between histone H3/H4 loss and Zeocin treatment. The same effect was observed using another source of DNA damage, ionizing radiation (γ-IR).

Despite being highly quantitative for protein abundance, mass spectrometry data does not distinguish between histone pools and nucleosomes, and it lacks positional information. To investigate whether entire nucleosomes were lost following DNA damage globally or at specific genomic loci, we performed genome-wide nucleosome mapping. First, the inventors found that the positioning of nucleosomes around the promoters of yeast genes changed little following damage induction. To assess global changes in nucleosome abundance, the inventors implemented internal standardization by mixing defined numbers of Candida glabrata cells with the experimental Saccharomyces cerevisiae cells prior to chromatin preparations. Normalization of the S. cerevisiae reads with respect to the C. glabrata reads showed that there was a drop in nucleosome occupancy both within promoters and across coding regions following Zeocin treatment. This effect was just as strong on a subset of 750 low expression genes as on highly transcribed genes, suggesting that transcription is unlikely to regulate or drive the reduction. Finally, the inventors found no preferential depletion specific structural elements such as centromeres or telomeres, arguing that the effect is widespread.

To determine the kinetics of histone reduction, the inventors used time-lapse live cell tracking of functional fluorescently labeled ectopic Histone H3 (H3-CFP) or control Htz1-mEos and Nup49-GFP, which labels the nuclear rim. the inventors used microfluidic chambers to trap cells and pulse-treated them for 1 h with Zeocin, generating roughly 4-7 DSBs per genome. Histone H3 degradation (˜30% compared to undamaged cells) occurred within 30 minutes of Zeocin exposure. Neither Nup49-GFP nor the Htz1-mEos control showed differential loss following DNA damage, suggesting that the induced histone degradation only targets core histones. Combined with the mass-spectroscopy and immunoblot data, these results suggested a rapid degradation of histones, rather than simply eviction from chromatin. Earlier, Gunjan et al. had shown that an excess of nonchromatin-bound histones is phosphorylated by the Rad53 checkpoint kinase, and then subsequently ubiquitinated and subject to proteasomal degradation (Gunjan and Verreault 2003). This prompted the inventors to test whether the proteasome inhibitor MG132 or mutation of the 26S proteasome (pre1-1, pre2-2) would suppress the loss of histones from chromatin. Consistent with proteasome involvement, both the inhibitor and the mutations in PRE1 and PRE2 genes suppressed the DNA damage-induced H3 or H4 degradation. Moreover, by synchronizing cells in G1, or releasing them into S phase prior to damage, the inventors found that degradation occurs in both phases of the cell cycle.

The inventors considered that the observed histone loss might be accentuated by impaired expression of histone genes, which are tightly regulated and show promoter-dependent upregulation in S phase. To eliminate this confounding factor, the inventors placed the H3 and H4 genes under the control of the galactose promoter in a strain in which both endogenous H3 and an active degradation of histones, and not simply a loss of new histone synthesis. Thus the observations uncover a novel facet of the DNA damage response mechanism through H4 copies were deleted (histone-shutdown strain). With constitutive H3/H4 expression (growth in media with low level galactose), the inventors found the same depletion effect following exposure to Zeocin as in cells with endogenous histone genes, arguing that DNA damage removes core histones from chromatin and degrades them. The loss of histones is rapid and so substantial (20-40%) that by 1 h, every third nucleosome could be removed from DNA. It is therefore likely that higher-order chromatin structure changes in response to DNA damage.

Damaged Chromatin Increases Mobility, Decompaction and Flexibility:

The increase in chromatin movement following DNA damage has been well documented, although the mechanisms leading to enhanced mobility remained elusive (Dion and Gasser 2013). To see if histone loss might be at the root of this phenomenon, the inventors examined the physical characteristics of yeast chromatin under the same conditions that triggered histone loss. Using improved imaging protocols, they monitored the volume of chromatin domains in three-dimensional (3D) space, the inherent flexibility of the nucleosome polymer and the physical movement of fluorescently tagged sites.

Previous studies in which chromatin mobility was quantified used low sampling rates during live cell imaging (Δt=1.5 sec) to determine the trajectory of a moving locus and the area explored (radius of constraint), e.g. Strecker, J. et al. (Strecker et al. 2016). However, such low time-resolved data yields little information on chromatin fiber compaction or flexibility. To resolve this, the inventors used a novel high-speed imaging technique (300 ms or 80 ms imaging intervals) with which they first confirmed that increased chromatin mobility can be monitored at a non-damaged site (MET10) in cells responding to widespread DNA damage. By applying an analysis based on polymer models to our high-speed imaging data (Amitai A., Seeber A. et al., under review), the inventors estimated biophysical parameters that predict both the expansion of chromatin (reflected by an increase in the anomalous exponent α) and the loss of constraining forces that limit chromatin movement (as seen by decrease in the spring constant K_{C}).

To examine whether the 3D volume of a defined chromatin domain was altered within the nucleus, the inventors used super-resolution microscopy coupled with subsequent machine-learning and 3D pixel classification analysis. Using this technique, they measured the change in volume of TetR-mCherry tagged chromosomal loci (chromatin expansion) in cells fixed 30 min after exposure to different amounts of Zeocin. Indeed, they scored a dose-dependent decompaction of S phase chromatin: 3D TetR-mCherry foci volumes expanded with increased amounts of damage. The second prediction from the polymer modeling of locus dynamics was that the flexibility of the chromatin fiber would be enhanced after DNA damage. Thus, the inventors monitored chromatin flexibility with confocal microscopy and measured the 3D distances between two differentially labeled genomic loci positioned on the same chromosome arm. They used two independent sets of loci spaced at genomic distances of either 320 kbp on Chr XIV or 50 kbp on Chr III. For the first set, they synchronized cells, fixed them before or after Zeocin treatment and calculated the average of all distances measured between the lacI-GFP and TetR-mRFP fluorescently tagged loci. The inventors find that after DNA damage, the average inter-spot distance increases significantly both in G1-(0.97-1.2 μm) and S-phase cells (0.99-1.12 μm). For the second set of data, a similar approach was taken but they measured the inter-distance between CFP-lacI and TetR-mRFP tagged foci on Chr III in real time. In all cases they included Rad52-GFP and ensured that there was no overlap of Rad52-GFP with either of the other two fluorescent signals, assuring that the measured changes do not arise from effects linked to local DNA repair events. Analysis of relative mean squared distance changes and the average of all measured inter-distances reveals a robust increase in inter-spot dynamics and distances following Zeocin treatment. These data are consistent with a model in which damage-triggered histone degradation reduces the amount of nucleosomal constraints within the chromatin fiber, causing chromatin to expand. The enhanced physical dynamics would be a reflection of increased flexibility.

Histone Abundance Dictates Chromatin Movement and Decompaction:

To confirm that increased chromatin mobility and decompaction arise as a consequence of histone loss, the inventors made use of a histone-shutdown strain that expresses H3 and H4 under the control of the GALL-10 promoter which is susceptible to media-controlled repression as well as induction. After 1 h in galactose, they released α-factor arrested cells bearing this shutdown construct into raffinose-containing medium. Depending on the concentration of raffinose, they observed reduced GAL1-10-driven expression, lowering histone H3 and H4 levels in a controlled manner by 25-40% within an hour. This artificial reduction of histones did not cause DNA damage checkpoint activation, even when levels were reduced extensively. Using the appropriate galactose:raffinose mixture, however, the inventors could reduce histone levels in a controlled manner, even in the absence of damage, after which they monitored both chromatin decompaction and a striking increase of chromatin mobility, measured at the MGS1 locus after 1 h on the defined medium.

To further validate these findings, the inventors made use of a mutant bearing deletions of both high-mobility group protein one (HMGB1) orthologues NHP6A and NHP6B (nhp6aΔnhp6bΔ, for simplicity called nhp6Δ), which was previously described as having reduced levels of core histone proteins (Celona et al. 2011) Here, they show that nhp6A does not trigger endogenous damage checkpoints, and has neither an altered FACS distribution nor Rad53 activation, yet by tracking chromatin mobility with the high-speed imaging regime they find that the mobility of two labeled foci, MET10 and PES4, is significantly enhanced in nhp6Δ cells. High resolution time-lapse imaging of the GFP-LacI-tagged PES4 or the TetR-mCherry-tagged MET10 locus further confirms an increase in chromatin flexibility which is reflected by a decrease in the spring constant K_{C}, and a positive trend in the anomalous exponent α. Finally, using super-resolution microscopy the inventors monitored an increase in 3D volume of the TetR-mCherry labeled MET10 locus in nhp6Δ cells, which was more pronounced in an asynchronous culture, for unknown reasons. Combined with the effects observed in the histone shutdown strain, these manipulations argue for a direct link between histone levels and chromatin movement.

Histone Loss is Checkpoint and INO80-C Dependent and Modulates Recombination Efficiency:

DNA damage activates the central DDC kinase Mec1 (ATR) which initiates a widespread phosphorylation cascade leading to a global damage response and cell-cycle arrest. Additionally, repair proteins such as Mrell, Exo1, Rad51 and Rad52 act locally on DNA to mediate resection and preparation for either repair by homologous recombination or end-joining. Among Mec1 targets are the downstream effector kinase Rad53 (CHK2)² and multiple subunits of the INO80-C remodeler (Poli et al. 2016). Since both INO80-C and DDC proteins were implicated in a general increase in chromatin mobility in response to DNA damage, the inventors hypothesized that these factors may also regulate histone loss, which we find can trigger enhanced chromatin mobility.

Using quantitative immunoblotting, they found that strains lacking checkpoint kinases Mec1 or Rad53 completely abolished histone degradation after Zeocin treatment. More strikingly, the same dependency was observed for strains deleted for INO80-C subunits Arp8, Ies4 or Arp5 which do not participate in the DDC, but remodel nucleosomes. Importantly, histone loss occurred independently of Rad51 and Exo1 showing that local repair events are not necessary for the DDC-triggered degradation of histones. The inventors further confirmed this with two other assays: H3-CFP fluorescence monitoring over time and super-resolution microscopy of tagged locus 3D volumes. In all cases they find that histone loss and chromatin expansion required the Mec1-mediated checkpoint and intact INO80-C: no histone loss or chromatin expansion is seen in mec1Δsml1Δ and rad53A nor in arp8Δ), while cells bearing sml1Δ (a control for the mec1Δsml1Δ) and rad51Δ behaved like their wild-type counterparts in response to damage.

The main role of the DDC kinase Mec1/ATR is to trigger a cell-wide stress response that helps the cell cope with DNA damage. This appears to be, at least in part, mediated by the remodeler INO80-C(Poli et al. 2016). The importance of chromatin-remodeling in histone degradation, is not entirely surprising, given that Ino80 was recently shown to interact with Cdc48, an AAA+ATPase involved in proteasome-dependent protein degradation (Lafon et al. 2015). Moreover, both Mec1 and INO80-C are linked to RNA Pol II eviction at sites of replication fork-transcription collision. Thus, these genetic dependencies further validate our model that histone degradation and chromatin expansion are the key phenomena underlying damage-enhanced chromatin movement. The data further suggest that a failure to degrade histones might impair the access of repair proteins to chromatin, giving an explanation for previously observed repair deficiencies in these mutants (van Attikum et al. 2007, Chen et al. 2012).

To examine the functional relevance of the observed reduction in nucleosome occupancy triggered by DNA damage, and to test the hypothesis that nucleosome reduction facilitates homologous recombination and thus DNA repair, the inventors made use of a recombination assay that monitors the integration rates of two different URA3 cassettes (800 bp homology or 82 bp homology) at two independent loci (MGS1 and URA3). In otherwise isogenic haploid strains, they impaired INO80-C activity by disrupting its nucleosome-binding subunit Arp8 (arp8Δ) or deleted both NHP6 genes, to reduce nucleosome levels genome-wide (Celona et al. 2011). Consistent with previously reported recombination defects in arp8A (Lisby et al. 2004, van Attikum et al. 2007), the inventors see reduced recombination rates in this mutant, while rates were significantly increased in the nhp6Δstrain. Interestingly, it had been shown that deletion of the Histone H3-H4 gene copy 2 (HHT2-HHF2) can confer resistance to DNA damaging agents and restore the viability of DDC mutants under stress conditions (Liang et al. 2012). Thus, the inventors hypothesized that artificially lowering histone levels by Nhp6 removal might rescue arp84 sensitivity and even increase the fitness of wild-type cells under damaging conditions. Using a recovery assay that scores cell survival after a 1 h treatment with increasing amounts of Zeocin, they found that nhp6Δcells recover better from acute DNA damage than a wild-type strain, and that lowering nucleosome occupancy by deleting NHP6 partially rescues the Zeocin sensitivity of an arp8Δstrain.

The observation that increased recombination rates in nhp6Δcells stem from changes in nucleosome occupancy, prompted the inventors to test whether gene targeting rates could also be increased by other approaches that reduce histone levels. Hence, they used the same recombination assay in their histone-shutdown strain and followed the integration of two different hygromycin-resistance markers either at ATG2 or MGS1. This was done directly after a 2 h incubation in raffinose-containing medium (raffinose only or a defined 1:20 galactose:raffinose mixture) which reduces histone H3 and H4 levels. Consistent with the nhp64 experiment, they found that a reduction of histone levels by means of transcriptional repression significantly enhances the integration rates of both ATG2::hygro and MGS1::hygro PCR products.

### REFERENCES

Celona, B., Weiner, A., Di Felice, F., Mancuso, F. M., Cesarini, E., Rossi, R. L., Gregory, L., Baban, D., Rossetti, G., Grianti, P., Pagani, M., Bonaldi, T., Ragoussis, J., Friedman, N., Camilloni, G., Bianchi, M. E. and Agresti, A. (2011) ‘Substantial histone reduction modulates genomewide nucleosomal occupancy and global transcriptional output’, PLoS Biol, 9(6), e1001086.
Chen, X., Cui, D., Papusha, A., Zhang, X., Chu, C. D., Tang, J., Chen, K., Pan, X. and ha, G. (2012) ‘The Fun30 nucleosome remodeller promotes resection of DNA double-strand break ends’, Nature, 489(7417), 576-80.
Chevalier, B. S., Kortemme, T., Chadsey, M. S., Baker, D., Monnat, R. J. and Stoddard, B. L. (2002) ‘Design, activity, and structure of a highly specific artificial endonuclease’, Mol Cell, 10(4), 895-905.
Chevalier, B. S. and Stoddard, B. L. (2001) ‘Homing endonucleases: structural and functional insight into the catalysts of intron/intein mobility’, Nucleic Acids Res, 29(18), 3757-74.
Choulika, A., Perrin, A., Dujon, B. and Nicolas, J. F. (1995) ‘Induction of homologous recombination in mammalian chromosomes by using the I-SceI system of Saccharomyces cerevisiae’, Mol Cell Biol, 15(4), 1968-73.
Dion, V. and Gasser, S. M. (2013) ‘Chromatin movement in the maintenance of genome stability’, Cell, 152(6), 1355-64.
Doetschman, T., Maeda, N. and Smithies, 0. (1988) ‘Targeted mutation of the Hprt gene in mouse embryonic stem cells’, Proc Natl Acad Sci USA, 85(22), 8583-7.
Gao, F., Shen, X. Z., Jiang, F., Wu, Y. and Han, C. (2016) ‘DNA-guided genome editing using the Natronobacterium gregoryi Argonaute’, Nat Biotechnol, 34(7), 768-73.
Gunjan, A. and Verreault, A. (2003) ‘A Rad53 kinase-dependent surveillance mechanism that regulates histone protein levels in S. cerevisiae’, Cell, 115(5), 537-49.
Haase, S. B. and Lew, D. J. (1997) ‘Flow cytometric analysis of DNA content in budding yeast’, Methods Enzymol, 283, 322-32.
Haft, D. H., Selengut, J., Mongodin, E. F. and Nelson, K. E. (2005) ‘A guild of 45 CRISPR-associated (Cas) protein families and multiple CRISPR/Cas subtypes exist in prokaryotic genomes’, PLoS Comput Biol, 1(6), e60.
Jasin, M. (1996) ‘Genetic manipulation of genomes with rare-cutting endonucleases’, Trends Genet, 12(6), 224-8.
Jinek, M., Chylinski, K., Fonfara, I., Hauer, M., Doudna, J. A. and Charpentier, E. (2012) ‘A programmable dual-RNA-guided DNA endonuclease in adaptive bacterial immunity’, Science, 337(6096), 816-21.
Kalish, J. M. and Glazer, P. M. (2005) ‘Targeted genome modification via triple helix formation’, Ann NY Acad Sci, 1058, 151-61.
Lafon, A., Taranum, S., Pietrocola, F., Dingli, F., Loew, D., Brahma, S., Bartholomew, B. and Papamichos-Chronakis, M. (2015) ‘IN080 Chromatin Remodeler Facilitates Release of RNA Polymerase II from Chromatin for Ubiquitin-Mediated Proteasomal Degradation’, Mol Cell, 60(5), 784-96.
Lange, S. S., Mitchell, D. L. and Vasquez, K. M. (2008) ‘High mobility group protein B1 enhances DNA repair and chromatin modification after DNA damage’, Proc Natl Acad Sci USA, 105(30), 10320-5.
Liang, D., Burkhart, S. L., Singh, R. K., Kabbaj, M. H. and Gunjan, A. (2012) ‘Histone dosage regulates DNA damage sensitivity in a checkpoint-independent manner by the homologous recombination pathway’, Nucleic Acids Res, 40(19), 9604-20.
Lieber, M. R. (2010) ‘The mechanism of double-strand DNA break repair by the nonhomologous DNA end-joining pathway’, Annu Rev Biochem, 79, 181-211.
Lisby, M., Barlow, J. H., Burgess, R. C. and Rothstein, R. (2004) ‘Choreography of the DNA damage response: spatiotemporal relationships among checkpoint and repair proteins’, Cell, 118(6), 699-713.
Liu, Y., Nairn, R. S. and Vasquez, K. M. (2009) ‘Targeted gene conversion induced by triplex-directed psoralen interstrand crosslinks in mammalian cells’, Nucleic Acids Res, 37(19), 6378-88.
Majumdar, A., Muniandy, P. A., Liu, J., Liu, J. L., Liu, S. T., Cuenoud, B. and Seidman, M. M. (2008) ‘Targeted gene knock in and sequence modulation mediated by a psoralen-linked triplex-forming oligonucleotide’, J Biol Chem, 283(17), 11244-52.
Makarova, K. S., Grishin, N. V., Shabalina, S. A., Wolf, Y. I. and Koonin, E. V. (2006) ‘A putative RNA-interference-based immune system in prokaryotes: computational analysis of the predicted enzymatic machinery, functional analogies with eukaryotic RNAi, and hypothetical mechanisms of action’, Biol Direct, 1,7.
Mansour, S. L., Thomas, K. R. and Capecchi, M. R. (1988) ‘Disruption of the proto-oncogene int-2 in mouse embryo-derived stem cells: a general strategy for targeting mutations to non-selectable genes’, Nature, 336(6197), 348-52.
Ohta, K., Shibata, T. and Nicolas, A. (1994) ‘Changes in chromatin structure at recombination initiation sites during yeast meiosis’, EMBO J, 13(23), 5754-63.
Orr-Weaver, T. L. and Szostak, J. W. (1983) ‘Yeast recombination: the association between double-strand gap repair and crossing-over’, Proc Natl Acad Sci USA, 80(14), 4417-21.
Orr-Weaver, T. L., Szostak, J. W. and Rothstein, R. J. (1981) ‘Yeast transformation: a model system for the study of recombination’, Proc Natl Acad Sci USA, 78(10), 6354-8.
Paques, F. and Haber, J. E. (1999) ‘Multiple pathways of recombination induced by double-strand breaks in Saccharomyces cerevisiae’, Microbiol Mol Biol Rev, 63(2), 349-404.
Pasero, P., Duncker, B. P., Schwob, E. and Gasser, S. M. (1999) ‘A role for the Cdc7 kinase regulatory subunit Dbf4p in the formation of initiation-competent origins of replication’, Genes Dev, 13(16), 2159-76.
Pingoud, A., Fuxreiter, M., Pingoud, V. and Wende, W. (2005) ‘Type II restriction endonucleases: structure and mechanism’, Cell Mol Life Sci, 62(6), 685-707.
Poli, J., Gerhold, C. B., Tosi, A., Hustedt, N., Seeber, A., Sack, R., Herzog, F., Pasero, P., Shimada, K., Hopfner, K. P. and Gasser, S. M. (2016) ‘Mec1, INO80, and the PAF1 complex cooperate to limit transcription replication conflicts through RNAPII removal during replication stress’, Genes Dev, 30(3), 337-54.
Price, B. D. and D'Andrea, A. D. (2013) ‘Chromatin remodeling at DNA double-strand breaks’, Cell, 152(6), 1344-54.
Roberts, R. J., Vincze, T., Posfai, J. and Macelis, D. (2003) ‘REBASE: restriction enzymes and methyltransferases’, Nucleic Acids Res, 31(1), 418-20.
Rouet, P., Smih, F. and Jasin, M. (1994) ‘Expression of a site-specific endonuclease stimulates homologous recombination in mammalian cells’, Proc Natl Acad Sci USA, 91(13), 6064-8.
Seeber, A., Dion, V. and Gasser, S. M. (2013) ‘Checkpoint kinases and the INO80 nucleosome remodeling complex enhance global chromatin mobility in response to DNA damage’, Genes Dev, 27(18), 1999-2008.
Strecker, J., Gupta, G. D., Zhang, W., Bashkurov, M., Landry, M. C., Pelletier, L. and Durocher, D. (2016) ‘DNA damage signalling targets the kinetochore to promote chromatin mobility’, Nat Cell Biol, 18(3), 281-90.
Stros, M. (2010) ‘HMGB proteins: interactions with DNA and chromatin’, Biochim Biophys Acta, 1799(1-2), 101-13.
Sung, P. and Klein, H. (2006) ‘Mechanism of homologous recombination: mediators and helicases take on regulatory functions’, Nat Rev Mol Cell Biol, 7(10), 739-50.
Szostak, J. W., Orr-Weaver, T. L., Rothstein, R. J. and Stahl, F. W. (1983) ‘The double-strand-break repair model for recombination’, Cell, 33(1), 25-35.
Travers, A. A. (2003) ‘Priming the nucleosome: a role for HMGB proteins?’, EMBO Rep, 4(2), 131-6.
van Attikum, H., Fritsch, O. and Gasser, S. M. (2007) ‘Distinct roles for SWR1 and INO80 chromatin remodeling complexes at chromosomal double-strand breaks’, EMBO J, 26(18), 4113-25.
Wiechens, N., Singh, V., Gkikopoulos, T., Schofield, P., Rocha, S. and Owen-Hughes, T. (2016) ‘The Chromatin Remodelling Enzymes SNF2H and SNF2L Position Nucleosomes adjacent to CTCF and Other Transcription Factors’, PLoS Genet, 12(3), e1005940.

1. A method for increasing the frequency of insertion of a nucleotide sequence of interest in a specific chromosomal site within the genome of a eukaryotic cell, said method comprising the step of modifying the state of chromatin in said cell.

2. The method according to claim 1, wherein the state of chromatin in said cell is modified by lowering nucleosome occupancy.

3. The method according to claim 2, wherein nucleosome occupancy is lowered transiently.

4. The method according to claim 1, wherein said chromatin state is altered by reducing nucleosomal occupancy by modulating the activity of high-mobility group box (HMGB) proteins.

5. The method according to claim 4, wherein HMGB proteins are inhibited by an agent selected from the group consisting of Glycyrrhizin, Tanshinone IIA, Epigallocatechin-3-gallate, Quercetin, Lycopene, nafamostat mesilate, gabexate mesilate, ethyl pyruvate, carbenoxolone, antibodies and Oligonucleotide (ODN)-based inhibitors of HMGB1.

6. The method according to claim 1, further comprising introducing into said eukaryotic cell a composition comprising a cassette comprising said nucleotide sequence of interest and at least a first region having sequence identity with said chromosomal site, wherein said nucleotide sequence of interest is inserted into the target site.

7. The method according to claim 6, wherein the insertion event is by a homologous recombination or non-homologous end-joining.

8. The method according to claim 6, wherein the composition further comprises an agent introducing double-strand breaks (DSB) within the genome.

9. The method according to claim 8, wherein the composition comprises a DSB agent selected from the group comprising consisting of Transcription activator-like effector nucleases (TALEN), zinc-finger nucleases (ZFN), CRISPR/Cas9, CRISPR/nickase, megaendonucleases, NgAgo and chimeric nucleases.

10. The method according to claim 8, wherein the double-strand break is at a specific chromosomal site within the genome of a eukaryotic cell.

11. A kit comprising an agent lowering nucleosome occupancy in eukaryotic cells and a targeting cassette.

12. The kit of claim 11 further comprising an agent inducing double strand breaks in eukaryotic cells.

13. The kit of claim 11 or 12, wherein said agent lowering nucleosome occupancy in eukaryotic cells is an HMGB1 inhibitor.

14. The kit of claim 13 wherein said HMGB1 inhibitor is selected from the group consisting of Glycyrrhizin, Tanshinone IIA, Epigallocatechin-3-gallate, Quercetin, Lycopene, nafamostat mesilate, gabexate mesilate, ethyl pyruvate, carbenoxolone, antibodies and Oligonucleotide (ODN)-based inhibitors of HMGB1.

15. The kit of claim 12, wherein said agent inducing double strand breaks in eukaryotic cells is selected from the group consisting of Transcription activator-like effector nucleases (TALEN), zinc-finger nucleases (ZFN), CRISPR/Cas9, CRISPR/nickase, megaendonucleases, NgAgo and chimeric nucleases.
